(19) United States
(12) Patent Application Publication
(10) Pub. No.: US 2020/0315602 A1
(43) Pub. Date: Oct. 8, 2020

(54) Retractor Blade Including A Pair Of Clips

(71) Applicant: K2M, Inc., Leeburg, VA (US)

(72) Inventors: Patrick Melton, Seattle, WA (US); Michael Prosser, Herndon, VA (US)

(73) Assignee: K2M, Inc., Leesburg, VA (US)

(21) Appl. No.: 16/483,300

(22) PCT Filed: Feb. 5, 2018
(86) PCT No.: PCT/US2018/016827

§ 371 Date: Aug. 2, 2019

Related U.S. Application Data
(60) Provisional application No. 62/454,107, filed on Feb. 3, 2017.

(51) Int. Cl. A61B 17/02; A61B 17/70

(52) U.S. Cl. CPC A61B 17/025; A61B 17/0206; A61B 2017/681; A61B 2017/0256; A61B 17/7001

(57) ABSTRACT

A retractor system includes a retractor device and a blade assembly. The retractor device includes a support and first and second arms. The retractor blade assembly includes first and second blades. Each of the first and second blades includes an elongate portion and a tubular portion. The elongate portion includes an engaging portion releasably coupled with the retractor device. The tubular portion extends distally from the elongate portion. The tubular portion defines a bore dimensioned to receive a housing of a bone screw. The tubular portion includes a clip deflectable radially outward. The clip is deflectable between a first state, in which, the clip is deflected radially outward such that the clip disengages the housing of the bone screw positioned in the tubular portion, and a second state, in which, the clip engages the housing of the bone screw positioned in the tubular portion.

### CROSS-REFERENCE TO RELATED APPLICATIONS

This application claims priority to, and the benefit of, U.S. Provisional Patent Application Ser. No. 62/454,107, filed on Feb. 3, 2017, the entire contents of which are incorporated by reference herein.

### BACKGROUND

### Technical Field

The present disclosure relates to a device for spinal surgery and, more particularly, to a device and a method of distracting vertebral bodies.

### Background of Related Art

There has been considerable development of retractor devices that are adapted for use in less invasive procedures. Many of the recent developments are based on traditional types of retractor devices for open procedures, predominantly table-mounted devices of various designs. These devices tend to be cumbersome and are not well adapted for use in small incisions. Standard hand-held retractor devices can be modified to fit the contours of these small incisions, but they require manual manipulation to maintain a desired placement, thereby occupying one hand of the clinician or requiring another person to assist the clinician during the procedure. Typical retractor devices are also positioned into the soft tissue and are levered back to hold the wound open, frequently requiring re-positioning if they dislodge, obstruct the clinician's view, or interfere with access to the surgical site.

In a spinal fusion, at least two vertebral bodies are rigidly connected using bone screws implanted into the respective vertebral bodies with a solid metal rod spanning the distance between the screws. The insertion of pedicle or facet screws is relatively straightforward and can be accomplished through a minimal incision. The difficulty arises upon the introduction of a length of rod into a very small incision with extremely limited access and visibility. The minimal incision should be maintained in an open and accessible condition, i.e., as wide as practicable, for introduction of the rod.

In order to be truly minimally invasive, a spinal fusion procedure should have a minimum number of small incisions and not require significant tissue and/or muscle retraction. Furthermore, an improved approach should encompass as many variations and applications as possible thereby allowing the clinician to adjust the procedure to accommodate the anatomy and surgical needs of the patient as presented. For instance, spinal fusions should not be limited to just one or two levels.

Therefore, a continuing need exists for a retractor device that works with current instruments to provide the retraction needed in a spinal procedure with an ease of use and without impairing a view of the surgical field.

### SUMMARY

In accordance with an embodiment of the present disclosure, a retractor system includes a retractor device and a retractor blade assembly. The retractor device includes a support, a first arm fixedly secured with the support, and a second arm movable on the support between a first position, in which, the first and second arms are approximated and a second position, in which, the first and second arms are spaced apart. The first and second arms define respective guide channels. The retractor blade assembly includes first and second blades. Each of the first and second blades includes an elongate portion and a tubular portion. The elongate portion includes an engaging portion having a connecting pin configured to be releasably received in the guide channel of one of the first and second arms of the retractor device. The tubular portion extends distally from the elongate portion. The tubular portion defines a bore dimensioned to receive a housing of a bone screw. The tubular portion includes a clip deflectable radially outward. The clip includes a first tab and a chamfer defining a gap therebetween. The gap is dimensioned to receive a lip of the housing of the bone screw. The clip is deflectable between a first state, in which, the clip is deflected radially outward such that the clip disengages the housing of the bone screw positioned in the tubular portion, and a second state, in which, the clip engages the housing of the bone screw positioned in the tubular portion.

In an embodiment, the connecting pins of the first and second blades may be configured to be slidably received in the respective guide channels of the first and second arms of the retractor device. Each connecting pin may have a circular cross-section.

In another embodiment, the tubular portion of the first or second blades may include an interior wall having at least a pair of protrusions extending along a length of the tubular portion to align and center the housing of the bone screw within the bore of the tubular portion.

In yet another embodiment, the pair of protrusions may be disposed proximal of a distal end of the tubular portion.

In still yet another embodiment, the tubular portion may define first and second slots opposing each other. The first and second slots may be dimensioned to receive a spinal rod therethrough. The first slot may extend along an entire length of the tubular portion. The second slot may have a first end defining an aperture and a closed second end having an arcuate profile. In another embodiment, the first end may be located distal of the second end.

In still yet another embodiment, the elongate portion may include first, second, and third faces that are non-planar. In an embodiment, the first, second, and third faces may define a generally concaved profile.

In yet another embodiment, the clip of the tubular portion may be biased radially inward.

In accordance with another embodiment of the present disclosure, a kit for a spinal surgery includes a bone screw and a retractor system. The bone screw includes a housing and a shaft extending from the housing. The housing includes a lip. The housing defines an annular groove and a slot configured to receive a spinal rod. The retractor system includes a retractor device and a retractor blade assembly. The retractor device includes a support, a first arm fixedly secured with the support, and a second arm movable on the support between a first position, in which, the first and second arms are approximated and a second position, in which, the first and second arms are spaced apart. The first and second arms define respective guide channels. The retractor blade assembly includes first and second blades. Each of the first and second blades includes an elongate portion and a tubular portion. The elongate portion includes an engaging portion including a connecting pin configured to be releasably received in the guide channel of one of the first and second arms. The tubular portion extends distally from the elongate portion. The tubular portion defines a bore dimensioned to receive the housing of the bone screw. The tubular portion includes a clip deflectable radially outward. The clip includes a first tab and a chamfer defining a gap therebetween. The gap is dimensioned to receive the lip of the housing of the bone screw. The chamfer is dimensioned to be received in the annular groove of the housing of the bone screw. The clip is deflectable between a first state, in which, the clip is deflected radially outward such that the clip disengages the housing of the bone screw positioned in the tubular portion, and a second state, in which, the clip engages the housing of the bone screw positioned in the tubular portion.

In an embodiment, connecting pins of the first and second blades may have non-circular cross-sections.

In another embodiment, the kit may further include a spinal rod configured to be received in the slot of the bone screw.

In yet another embodiment, the retractor device may further include a third arm movable in a direction transverse to a direction of displacement of the second blade of the retractor blade assembly.

In accordance with another aspect of the present disclosure, a method of distracting adjacent vertebral bodies includes inserting first and second bone screws into respective first and second vertebral bodies; positioning first and second blades of a retractor system over the respective bone screws such that clips of respective tubular portions of the first and second blades engage housings of the respective bone screws; securing first and second arms of a retractor device of the retractor system with connecting pins of the first and second blades of the retractor system; and displacing the second arm of the retractor device away from the first arm to distract the first and second vertebral bodies.

In an embodiment, the method may further include clearing a disc space located between the first and second vertebral bodies.

In another embodiment, the method may further include implanting a device into the cleared disc space.

In yet another embodiment, positioning the first and second blades may include positioning a lip of the housing of one of the bone screws in a space between a first tab and a chamfer of the clip of one of the tubular portions.

In still yet another embodiment, positioning the first and second blades may include positioning the chamfer of the clip of one of the tubular portions in an annular groove defined in the housing of one of the bone screws.

### BRIEF DESCRIPTION OF THE DRAWINGS

Various embodiments of the present disclosure are described hereinbelow with reference to the drawings, wherein:

FIG. 1 is a perspective view of a retractor system in accordance with an embodiment of the present disclosure illustrating use with vertebral bodies;

FIG. 2 is a perspective view of a first blade of the retractor system of FIG. 1;

FIG. 3 is a side view of the first blade of FIG. 2;

FIG. 4 is an exploded, perspective view of a second blade of the retractor system of FIG. 1 with parts separated;

FIG. 5 is a top view of the second blade of FIG. 4;

FIG. 6 is a partial cross-sectional view of the second blade of FIG. 4 cut along section line 6-6 of FIG. 5;

FIG. 7 is a perspective view of the first blade and a bone screw of the retractor system of FIG. 1;

FIG. 8 is a side perspective view of the first blade and the bone screw of FIG. 7;

FIG. 9 is a front view of the first blade and the bone screw of FIG. 7;

FIG. 10 is a partial cross-sectional view of the first blade and the bone screw of FIG. 9 cut along section line 10-10 of FIG. 9;

FIG. 11 is a perspective view of the first blade and the bone screw of FIG. 7 illustrating use with a vertebral body with a driver;

FIG. 12 is a perspective view of the first and second blades and the bone screws of the retractor system of FIG. 1 illustrating use with vertebral bodies;

FIG. 13 is a perspective view of the retractor system of FIG. 1 illustrating use with the vertebral bodies with a third blade added; and

FIG. 14 is a perspective view of a removal tool for use with the first and second retractor blades of the retractor system of FIG. 1.

### DETAILED DESCRIPTION

Embodiments of the present disclosure will now be described in detail with reference to the drawings, in which like reference numerals designate identical or corresponding elements in each of the several views. As used herein, the term “distal,” as is conventional, will refer to that portion of the instrument, apparatus, device or component thereof which is farther from the user while, the term “proximal,” will refer to that portion of the instrument, apparatus, device or component thereof which is closer to the user. In addition, the term “cephalad” is used in this application to indicate a direction toward a patient's head, while the term “caudad” indicates a direction toward the patient's feet. Further still, for the purposes of this application, the term “medial” indicates a direction toward the middle of the body of the patient, while the term “lateral” indicates a direction toward a side of the body of the patient, i.e., away from the middle of the body of the patient. The term “posterior” indicates a direction toward the patient's back, while the term “anterior” indicates a direction toward the patient's front. In the following description, well-known functions or constructions are not described in detail to avoid obscuring the present disclosure in unnecessary detail.

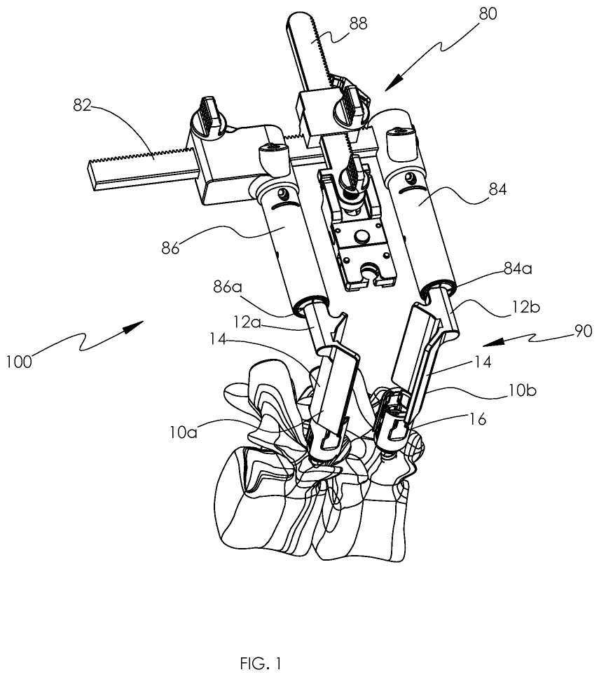

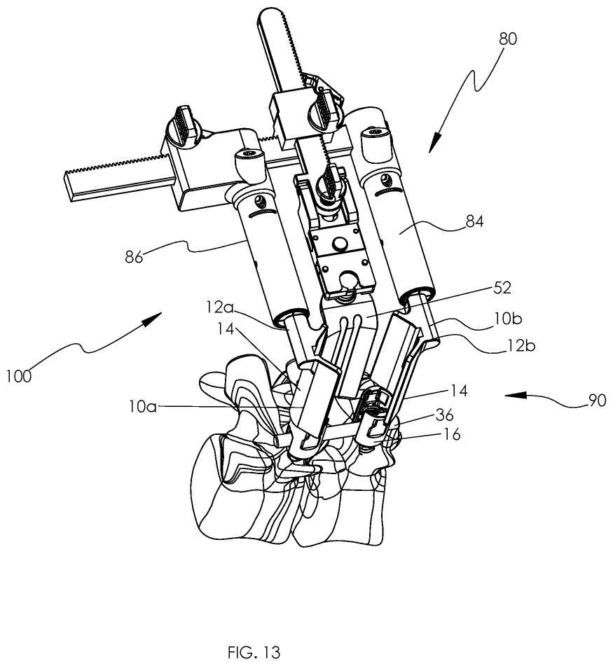

With reference to FIGS. 1-3, an embodiment of the present disclosure is shown generally as a retractor system 100 that is configured and adapted for use in a minimally invasive surgical procedure to access, for example, the cervical, thoracic, or lumbar vertebrae. For example, the retractor system 100 may be used in a discectomy for retracting soft tissue and distracting vertebral bodies. The retractor system 100 includes a retractor device 80 and a retractor blade assembly 90. The retractor device 80 includes a support 82, first and second arms 84, 86 configured to releasably support the retractor blade assembly 90, and an auxiliary arm 88 configured to releasably support a medial retractor blade 52 (FIG. 13).

The first arm 84 of the retractor system 80 is fixedly secured to the support 82, and the second arm 86 of the retractor system 80 is movably supported on the support 82. In particular, each of the first and second arms 84, 86 defines a guide channel 84a, 86a configured to be releasably coupled with the respective blades 10a, 10b of the retractor blade assembly 90. The second arm 86 may be selectively secured in a plurality of locations relative to the first arm 84 along the support 82. Movement of the second arm 86 along the support 82 transitions the retractor blade assembly 90 between an approximated position, in which, the blades 10a, 10b are adjacent to each other and, a spaced apart position, in which, the blades 10a, 10b are spaced apart to enable distraction of the vertebral bodies and retraction of tissue, as will be discussed below. Reference may be made to U.S. Pat. No. 8,449,463, the entire contents of which are incorporated herein by reference, for a detailed description of the construction and operation of a retractor device.

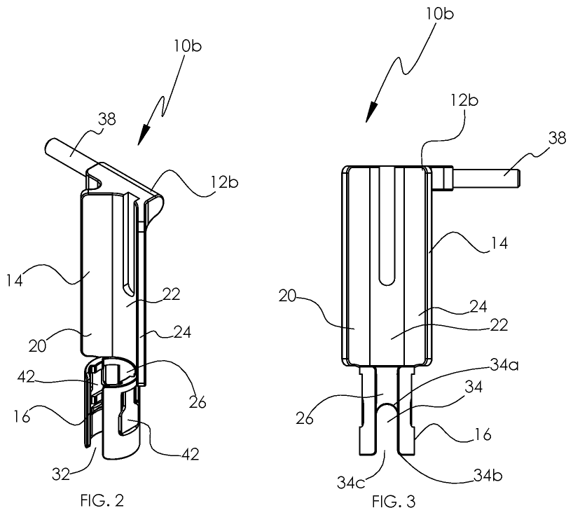

With reference now to FIGS. 2-4, the retractor blade assembly 90 (FIG. 1) includes the blades 10a, 10b that are substantially identical to each other. In particular, the blades 10a, 10b include respective engaging portions 12a, 12b that extend in opposite directions such that when the blades 10a, 10b are secured with vertebral bodies in opposing manner, the engaging portions 12a, 12b of the blades 10a, 10b extend in the same direction to engage the retractor device 80. Since the blades 10a, 10b are mirror images of each other, the blades 10a, 10b are described herein as being identical in order to avoid obscuring the present disclosure in unnecessary detail.

Each blade 10a, 10b includes an elongate portion 14 and a tubular portion 16 extending distally from the elongate portion 14. The elongate portion 14 includes the engaging portion 12a, 12b configured to receive a portion of a connecting pin 38 configured to be slidably received in a guide channel 84a, 86a (FIG. 1) of the first and second arms 84, 86 of the retractor device 80. While the connecting pins 38 are shown to have a circular cross-section, it is envisioned that the connecting pins 38 may include a non-circular cross-section to inhibit relative rotation between the blades 10a, 10b and the respective first and second arms 84, 86 of the retractor device 80. The connecting pins 38 may be releasably coupled with the engaging portion 12a, 12b of blade 10a, 10b and the retractor device 80. However, it is envisioned that the connecting pin 38 may be formed as a single construct with the engaging portion 12a, 12b.

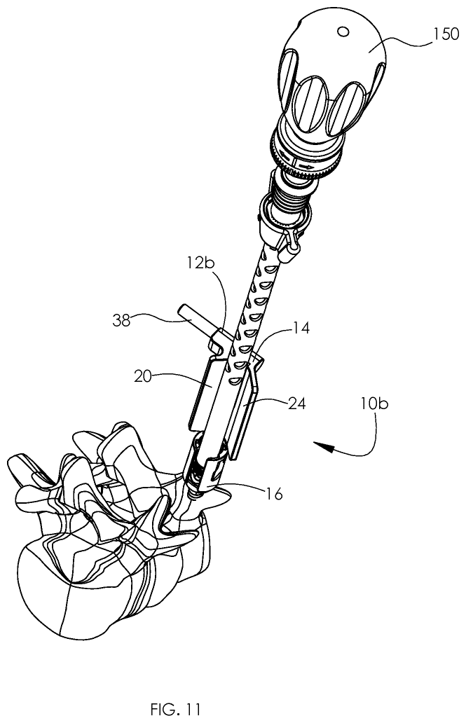

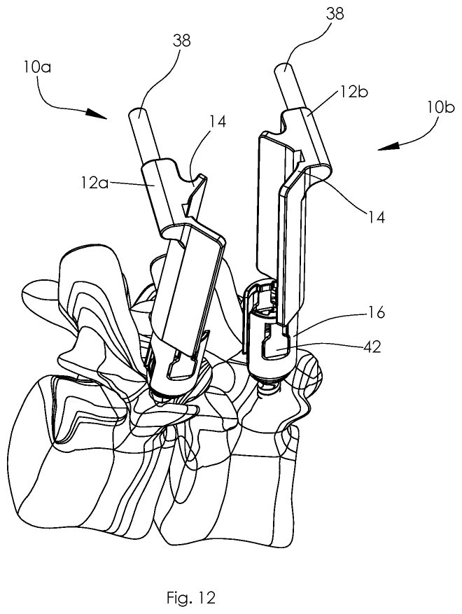

With continued reference to FIGS. 2-4, the elongate portion 14 includes a plurality of faces 20, 22, 24 that are angled relative to each other. The plurality of faces 20, 22, 24 of the elongate portion 14 defines a generally concaved profile or a groove configured to at least partially align with, i.e., partially surround, the circumference of the tubular portion 16. Under such a configuration, when the blades 10a, 10b are disposed in an opposed manner, the faces 20, 24 of the elongate portions 14 of the respective blades 10a, 10b are positioned towards each other to facilitate receipt of a driver 150 (FIG. 11) or to facilitate retraction of soft tissue to inhibit encroachment of tissue into the respective tubular portions 16. While three faces 20, 22, 24 are shown for each elongate portion 14, it is contemplated that a various number of faces may be utilized. It is further contemplated that a length and a width of the elongate portion 14 may be tailored to meet the needs of the particular surgical procedure.

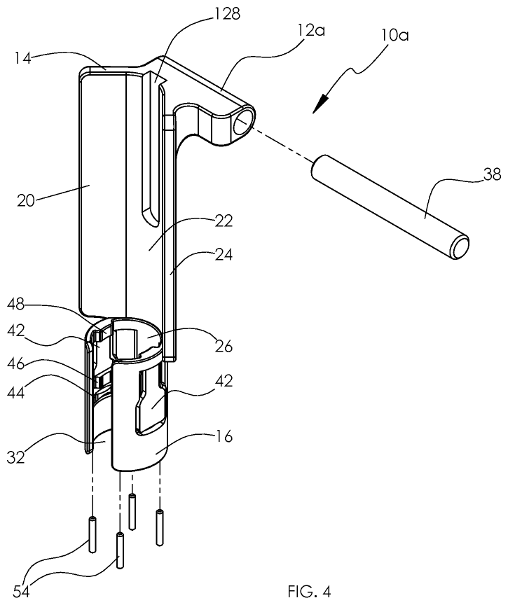

With reference to FIG. 4, the elongate portion 14 defines a channel 28 in the face 22. The channel 28 is dimensioned to receive, e.g., a ridge of a surgical instrument, to inhibit rotation of the blade 10a, 10b during insertion thereof

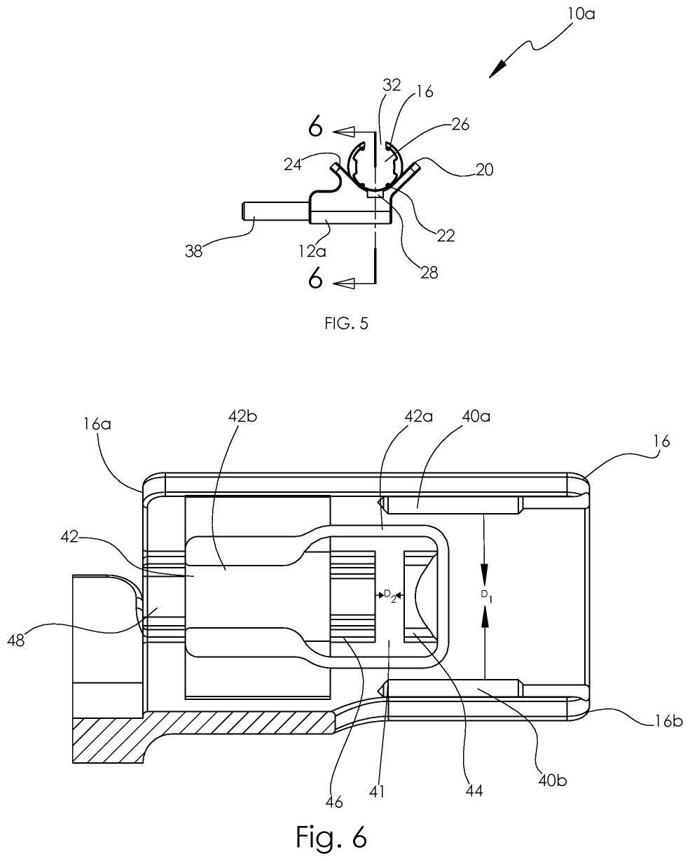

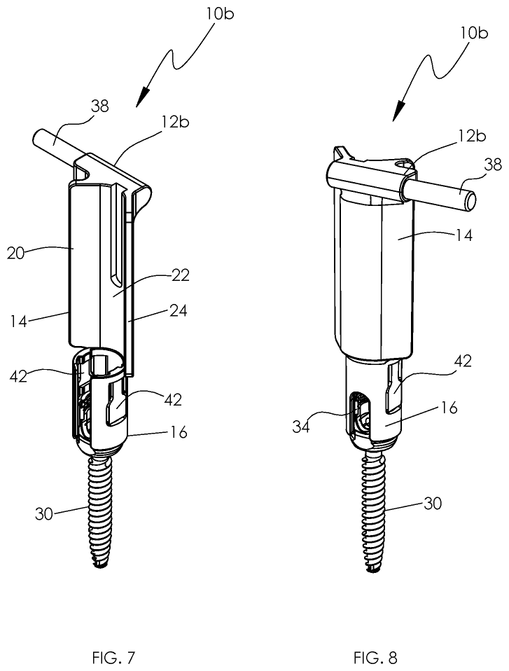

With reference now to FIGS. 3-6, the tubular portion 16 extends distally from the elongate portion 14. The tubular portion 16 defines a bore 26 dimensioned to receive the bone screw 30 (FIG. 7) therein. The tubular portion 16 further defines a first slot 32 extending along an entire length of the tubular portion 16, and a second slot 34 extending partially along the length of the tubular portion 16. The second slot 34 includes a proximal portion 34a having an arcuate wall configured to engage a spinal rod 36 (FIG. 13) and a distal portion 34b defining an aperture 34c. Under such a configuration, the spinal rod 36 may be placed in the bone screw 30 through the first and second slots 32, 34 of the tubular portion 16 when the bone screw 30 is disposed within the tubular portion 16.

With particular reference to FIGS. 4 and 6, the tubular portion 16 includes a plurality of pins 54 dimensioned to be received in a plurality of ducts 40a, 40b defined in an interior wall of the tubular portion 16. The pins 54 disposed in the ducts 40a, 40b at least partially protrude from the interior wall of the tubular portion 16 and are configured to align and center the bone screw 30 in the bore 26 of the tubular portion 16. The ducts 40a, 40b may diametrically oppose each other and a distance D₁ between the ducts 40a, 40b may be equivalent to a width of a housing 33 (FIG. 10) of the bone screw 30. The ducts 40a, 40b extend a portion of the length of the tubular portion 16. In particular, distal ends of the ducts 40a, 40b are proximal of the distal end 16b of the tubular portion 16 such that when the bone screw 30 is positioned in the distal portion 16b of the tubular portion 16, the bone screw 30 may freely rotate therein until the bone screw 30 is guided by the plurality of pins 54 into the bore 26 of the tubular portion 16. The pins 54 are configured to align and center the bone screw 30 within the tubular portion 16, while maintaining the bone screw 30 in place within the tubular portion 16. Alternatively, the interior wall of the tubular portion 16 may include a plurality of ridges or other raised surfaces integrally formed with the interior wall to achieve the desire alignment.

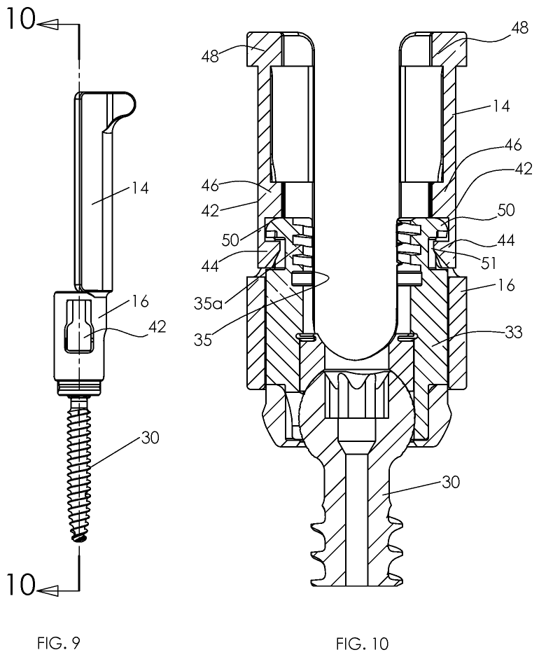

With reference now to FIGS. 6-9, the tubular portion 16 includes a pair of clips 42 diametrically opposing each other. The clips 42 are resilient. The clips 42 may be cut or machined from the tubular portion 16 to enable radial deflection and to provide a biasing force. Each clip 42 has a wider distal end 42a and a narrower proximal end 42b. A width of the distal end of the clip 42 may be equivalent to, or slightly smaller than, the width of the housing 33 (FIG. 10) of the bone screw 30. A width of the proximal end of the clip 42 may be narrower to assist in providing the biasing force to enable the clip 42 to detachably engage the housing 33 (FIG. 10) of the bone screw 30. The biasing force may vary depending on several variables such as a thickness of the clip 42 or material of construction. The biasing force may be reduced by reducing the thickness of the clip 42 by providing, e.g., a flat external surface, and the biasing force may be increased by increasing the thickness of the clip 42 by providing, e.g., a cylindrical external surface. Additionally, the biasing force may vary by altering the width of the proximal end 42b and the distal end 42a of the clip 42.

With particular reference to FIGS. 6 and 10, an inner surface 41 of the clip 42 includes a chamfer 44, a first tab 46, and a second tab 48. The chamfer 44 may be configured and dimensioned to engage an annular groove 51 defined in the housing 33 of the bone screw 30. The first tab 46 is proximal of the chamfer 44 and is separated from the chamfer 44 by a gap D₂, which may be equivalent to a thickness of a lip 50 of the housing 33 of the bone screw 30 such that the lip 50 of the housing 33 of the bone screw 30 may be received between the chamfer 44 and the first tab 46. Under such a configuration, the first tab 46 inhibits axial displacement of the housing 33 of the bone screw 30 towards the elongate portion 14.

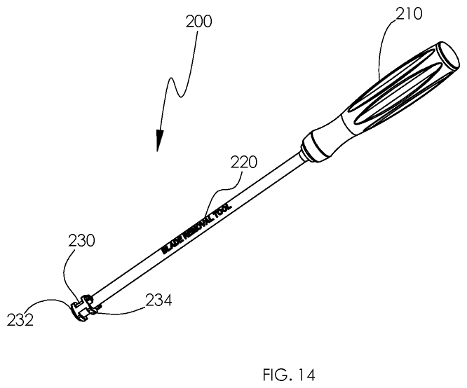

Additionally, the first tab 46 is configured to operatively engage a removal tool 200 (FIG. 14). In particular, the removal tool 200 includes a handle 210, a shaft 220 extending from the handle 210, and an engaging portion 230 including a first camming portion 232 and a second camming portion 234. The first camming portion 232 is configured to rotatably engage the first tab 46, which, in turn, deflects the clip 42 of the tubular portion 16 radially outward such that the clip 42 is disengaged from the housing 33 (FIG. 10) of the bone screw 30.

With continued reference to FIGS. 6 and 10, the second tab 48 of the clip 42 is disposed adjacent a proximal end 16a of the tubular portion 16. The second tab 48 is also configured to operatively engage the removal tool 200. In particular, the second tab 48 is configured to engage the second camming portion 234 of the removal tool 200. In particular, the removal tool 200 is rotatable between a first position, in which, the second camming portion 234 of the removal tool 200 is radially offset from the second tab 48 to enable axial displacement of the removal tool 200 relative to the tubular portion 16, and a second position, in which, the second camming portion 234 of the removal tool 200 is proximal of and in registration with the second tab 48.

In this manner, through rotation of the removal tool 200, the first camming portion 232 of the removal tool 200 engages the first tab 46 and deflects the clips 42 radially outward thereby disengaging the clip 42 from the housing 33 of the bone screw 30. At this time, the second camming portion 234 of the removal tool 200 is in registration with the second tab 48 such that when the clinician pulls the removal tool 200, i.e., away from the bone screw 30, the blade 10a, 10b is separated from the bone screw 30. In addition, the second tab 48 may also act as an alignment stop by inhibiting over-rotation of the removal tool 200.

In use, initially, the clinician prepares the vertebral bodies. For example, the clinician may optionally utilize an indicator pin. The clinician locates the center of a vertebral disc space and inserts the indicator pin (not shown) thereto. The indicator pin may serve as a center point in a medial-lateral direction and in the cephalad-caudal direction. A drill guide (not shown) may be utilized to locate insertion points for the bone screws 30. Specifically, the drill guide is inserted through the indicator pin such that the drill guide is in registration with the vertebral bodies. At this time, a drill (not shown) can be utilized to form holes in the vertebral bodies to receive the respective bone screws 30. With reference now to FIG. 11, after the bone screws 30 are fixed with the vertebral bodies, the blades 10a, 10b can be positioned over the housing 33 (FIG. 10) of the bone screws 30. In particular, the plurality of pins 54 facilitates alignment of the housing 33 within the bore 26 of the tubular portion 16. A downward force can be applied to the blade 10a, 10b by the clinician such that the diametrically opposing clips 42 on the tubular portion 16 engage the housing 33 of the bone screw 30. The lip 50 of the housing 33 (FIG. 10) of the bone screw 30 is received between the chamfer 44 and the first tab 46 and the chamfer 44 is received in the annular groove 51 of the bone screw 30. In this manner, the blade 10a, 10b is clipped-on or attached to the housing 33 (FIG. 10) of the bone screw 30. At this time, the clinician may insert a driver 150 into the bore 26 of the tubular portion 16 to further drive the bone screw 30 into the vertebral body.

As can be appreciated with reference to FIG. 12, once one of the blades 10a is secured with the bone screw 30 affixed to a vertebral body, same steps may be carried out to secure a second blade 10b with an adjacent vertebral body. At this time, the retractor device 80 may be attached to the blades 10a, 10b. With reference now to FIG. 13, the clinician at this time can selectively move the second arm 86 of the retractor device 80 in the cephalad-caudal direction relative to the first arm 84. In this manner, the clinician can distract the vertebral bodies for a desired surgical procedure. Optionally, the medial blade 52 may also be attached to the retractor device 80 in order to retract soft tissue around the blades 10a, 10b in the medial-lateral direction. At this time, the disc space can be cleared using standard techniques and an implant device (not shown) can then be inserted into the cleared disc space. Upon inserting the implant device into the cleared disc space, the second arm 86 may be moved towards the first arm 84 until the vertebral bodies engage the implanted device. It is also contemplated that, e.g., a cervical plate, may be utilized to inhibit additional movement of the vertebral bodies. Reference may be made to U.S. Pat. No. 9,017,409, the entire contents of which are incorporated herein by reference, for a detailed description of the construction and operation of an implant device.

The clinician may also utilize the spinal rod 36 in the housing 33 of the bone screws 30 through the first and second slots 32, 34 of the tubular portion 16. The spinal rod 36 may be secured with the bone screw 30 by a set screw (not shown) threadably engaging threads 35a (FIG. 10) on an inner wall 35 of the housing 33 of the bone screw 30.

Upon completion of the surgical procedures, the retractor system 100 may be removed from the surgical site. First, the retractor device 80 is detached from the connecting pins 38 of the engaging portion 12a, 12b of the blades 10a, 10b. Thereafter the removal tool 200 (FIG. 14) may be utilized to remove the blades 10a, 10b.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure.

Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

1. A retractor system comprising:
a retractor device including:
a support;
a first arm fixedly secured with the support; and
a second arm movable on the support between a first position, in which, the first and second arms are approximated and a second position, in which, the first and second arms are spaced apart, the first and second arms defining respective guide channels; and
a retractor blade assembly including first and second blades, each of the first and second blades including:
an elongate portion including an engaging portion having a connecting pin configured to be releasably received in the guide channel of one of the first and second arms of the retractor device; and
a tubular portion extending distally from the elongate portion, the tubular portion defining a bore dimensioned to receive a housing of a bone screw, the tubular portion including a clip deflectable radially outward, the clip including a first tab and a chamfer defining a gap therebetween, the gap dimensioned to receive a lip of the housing of the bone screw, wherein the clip is deflectable between a first state, in which, the clip is deflected radially outward such that the clip disengages the housing of the bone screw positioned in the tubular portion, and a second state, in which, the clip engages the housing of the bone screw positioned in the tubular portion.

2. The retractor system according to claim 1, wherein the connecting pins of the first and second blades are configured to be slidably received in the respective guide channels of the first and second arms of the retractor device.

3. The retractor system according to claim 1, wherein each connecting pin has a circular cross-section.

4. The retractor system according to claim 1, wherein the tubular portion of the first or second blades includes an interior wall having at least a pair of protrusions extending along a length of the tubular portion to align and center the housing of the bone screw within the bore of the tubular portion.

5. The retractor system according to claim 4, wherein the pair of protrusions is disposed proximal of a distal end of the tubular portion.

6. The retractor system according to claim 4, wherein the tubular portion defines first and second slots opposing each other, the first and second slots dimensioned to receive a spinal rod therethrough.

7. The retractor system according to claim 5, wherein the first slot extends along an entire length of the tubular portion.

8. The retractor system according to claim 5, wherein the second slot has a first end defining an aperture and a closed second end having an arcuate profile.

9. The retractor system according to claim 8, wherein the first end is located distal of the second end.

10. The retractor system according to claim 1, wherein the elongate portion includes first, second, and third faces that are non-planar.

11. The retractor system according to claim 10, wherein the first, second, and third faces define a generally concaved profile.

12. The retractor system according to claim 1, wherein the clip of the tubular portion is biased radially inward.

13. A kit for a spinal surgery comprising:
a bone screw including a housing and a shaft extending from the housing, the housing including a lip, the housing defining an annular groove and a slot configured to receive a spinal rod; and
the retractor system: of claim 1
wherein the chamfer is dimensioned to be received in the annular groove of the housing of the bone screw.

14. The kit according to claim 13, wherein the connecting pins of the first and second blades have non-circular cross-sections.

15. The kit according to claim 13, further comprising a spinal rod configured to be received in the slot of the bone screw.

16. The kit according to claim 15, wherein the tubular portion defines first and second slots opposing each other, the first and second slots dimensioned to receive the spinal rod therethrough.

17-18. (canceled)

19. The kit according to claim 13, wherein the elongate portion has a non-planar surface has a generally concaved profile.

20. The kit according to claim 13, wherein the retractor device further includes a third arm movable in a direction transverse to a direction of displacement of the second blade of the retractor blade assembly.

21. A method of distracting adjacent vertebral bodies comprising:
inserting first and second bone screws into respective first and second vertebral bodies;
positioning first and second blades of a retractor system over the respective bone screws such that clips of respective tubular portions of the first and second blades engage housings of the respective bone screws;
securing first and second arms of a retractor device of the retractor system with connecting pins of the first and second blades of the retractor system; and
displacing the second arm of the retractor device away from the first arm to distract the first and second vertebral bodies.

22-25. (canceled)

26. The retractor system according to claim 1, wherein the tubular portion and the elongate portion of the first blade extend along a first longitudinal axis, and the tubular portion and the elongate portion of the second blade extend along a second longitudinal axis, the first longitudinal axis being arranged to extend proximally from a first bone screw when the clip of the tubular portion of the first blade is in engagement with the housing of the first bone screw, and the second longitudinal axis being arranged to extend proximally from a second bone screw when the clip of the tubular portion of the second blade is in engagement with the housing of the second bone screw, and wherein, when clip of the tubular portion of the respective first and second blade is in engagement with the housing of the respective first and second bone screw, each of the respective tubular portions extends proximally along the respective first and second longitudinal axis to a location that is closer to the respective first and second bone screw than a proximal-most end of the respective elongate portion.
